(19) United States
(12) Plant Patent Application Publication
(10) Pub. No.: US 2002/0188977 P1
(43) Pub. Date: Dec. 12, 2002

(54) Chrysanthemum plant named 'GEDI ONE CEL'

(76) Inventor: Dirk Pieters, Staden (BE)

Correspondence Address: KNOBBE MARTENS OLSON & BEAR LLP, 2040 MAIN STREET, FOURTEENTH FLOOR, IRVINE, CA, 92614, US

(21) Appl. No.: 09/873,959

(22) Filed: Jun. 4, 2001

(51) Int. Cl. A01H 5/00

(52) U.S. Cl. PLT/288

(57) ABSTRACT

A new and distinct Chrysanthemum plant variety characterized by a decorative type inflorescence, consistent flowering response to short days, blooming consistently after 49 days of short day length, free branching habit, white ray florets, very uniform round growth habit, and a large quantity of blooms per flowering branch.

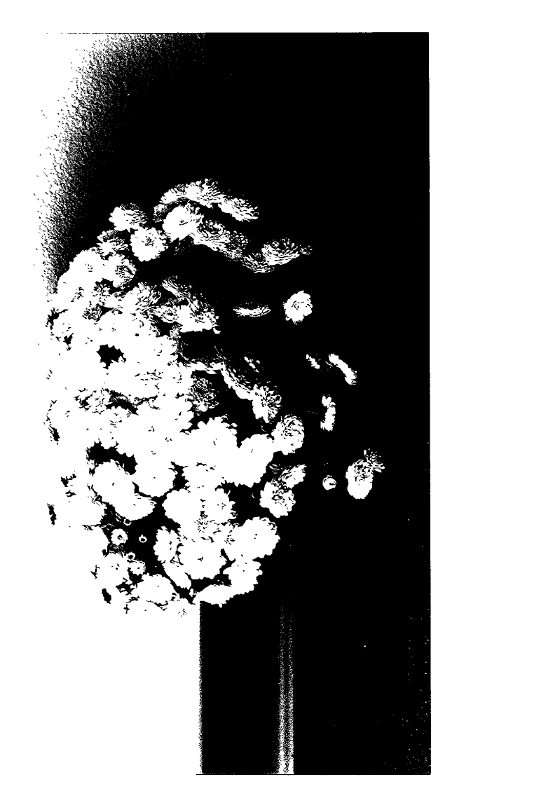

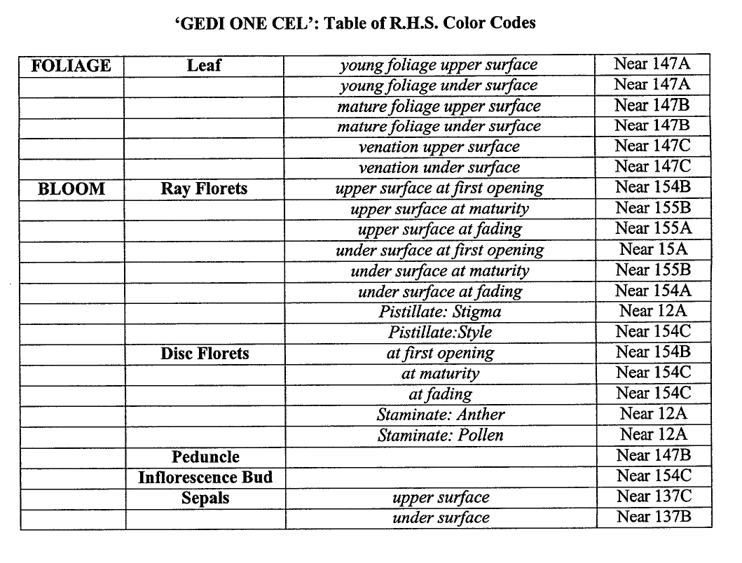

### BACKGROUND OF THE INVENTION

The present invention relates to a new and distinct cultivar of Chrysanthemum plant, botanically known as Dendranthema morifolium and herein after referred to by the cultivar name ‘GEDI ONE CEL.’

This new cultivar originated from hybridization of a female parent Chrysanthemum cultivar ‘Pinos’ (U.S. Plant Pat. No. 10,208) with a male parent Chrysanthemum cultivar ‘GEDI WO’ (U.S. Plant patent application Ser. No. 09/245,234). The new cultivar was discovered by the inventor in October 1997.

Asexual reproduction of the new cultivar by apical tip cutting and meristem tissue culture was performed in Oxnard, Calif. and has shown that the unique features of this new cultivar are stable and reproduced true to type on successive generations.

### SUMMARY OF THE INVENTION

The cultivar ‘GEDI ONE CEL’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘GEDI ONE CEL.’ These characteristics in combination distinguish ‘GEDI ONE CEL’ as a new and distinct Chrysanthemum:

1. Decorative type inflorescence;

2. Consistent flowering response to short days, blooming consistently after 49 days of short day length;

3. Free branching habit;

4. White ray florets;

5. Very uniform round growth habit; and

6. Large quantity of blooms per flowering branch.

Plants of the new cultivar are similar to plants of the parent cultivar, ‘Pinos,’ in most horticultural characteristics, however plants of the new cultivar differed from plants of the cultivar ‘Pinos’ in the following characteristics:

1. The new cultivar has more blooms per flowering branch;

2. The new cultivar has different color ray florets;

3. The new cultivar has a wider diameter bloom; and

4. The new cultivar is taller and wider.

Plants of the new cultivar are similar to plants of the parent cultivar, ‘GEDI WO,’ in most horticultural characteristics, however plants of the new cultivar differed from plants of the cultivar ‘GEDI WO’ in the following characteristics:

1. The new cultivar has a different flower type;

2. The new cultivar is taller and wider; and

3. The new cultivar has a wider diameter bloom.

The new cultivar can be compared to the Chrysanthemum cultivar ‘Odysseus’ (disclosed in Plant Pat. No. 11,637). However plants of the new Chrysanthemum differed from the plants of the cultivar ‘Odysseus’ in the following characteristics:

1. The new cultivar naturally blooms 2 weeks later;

2. The new cultivar has a more flexible plant habit;

3. The new cultivar has a more round plant shape and greater branching; and

4. The new cultivar has more blooms per flowering branch.

### BRIEF DESCRIPTION OF THE PHOTOGRAPHS

The photograph shows a plant of ‘GEDI ONE CEL’ grown in a 6-inch container. A single cutting was used in the container.

### DETAILED BOTANICAL DESCRIPTION

In the following description, color references are made to The Royal Horticultural Society Colour Chart except where general terms of ordinary dictionary significance are used. The following observations and measurements describe plants grown in a commercial filed on Oxnard, Calif. in the month of September 2000. The growing conditions range from 60° to 65° F. at night to 70° to 85° during the day. Measurements and numerical values represent averages of typical flowering types.

Botanical classification: Dendranthema morifolium cultivar ‘GEDI ONE CEL’

Commercial classification: Garden-type Chrysanthemum

Parentage: Hybridization of female parent Chrysanthemum cultivar ‘Pinos’ (U.S. Plant Pat. No. 10,208) with male parent Chrysanthemum cultivar ‘GEDI WO’ (U.S. Plant patent application Ser. N. 09/245,234)

### Propagation

Time to Rooting: 7 to 14 days at approximately 21° C.

Rooting habit: Fine, fibrous

### Plant

Growth Habit: Mounding herbaceous perennial

Height: Approximately 20 cm

Growth rate: Moderate

Branching Characteristics: Free Branching

Length of Lateral Branches: Approximately 17 cm

Number of leaves per lateral branch: Approximately 19

### Foliage

Leaf:

Arrangement.—Alternate.

Average length.—Approximately 5.5 cm.

Average width.—Approximately 3.5 cm

Shape of blade.—Ovate.

Apex.—Acuminate.

Base.—Attenuate.

Attachment.—Stalked.

Margin.—Serrate.

Texture of top surface.—Lightly pubescent.

Texture of bottom surface.—Lightly pubescent.

Color.—young foliage upper side: Near 147A young foliage under side: Near 147A mature foliage upper side: Near 147B mature foliage under side: Near 147B venation upper side: Near 147C venation under side: Near 147C.

Petiole:

Average length.—Approximately 1.5 cm

### Bloom

Inflorescence:

Flowering habit.—Induced by darkness period greater than 13.5 hours, approximately 49 days of appropriate day length required to induce and develop blooms

Inflorescence form.—Decorative

Natural flowering season.—Approximately the last week in September.

Number of inflorescences per stem.—Approximately 13.

Inflorescence diameter.—Approximately 3.3 cm.

Inflorescence depth.—Approximately 1.0 cm.

Inflorescence longevity on plant: Approximately 26-29 days

Persistence.—Persistent

Ray Florets:

Appearance.—Matte

Texture.—Shiny

Average number per flower.—Approximately 162

Shape.—Oblanceolate

Margin.—Entire

Apex.—Obtuse

Length.—Approximately 1.4 cm

Width.—Approximately 0.6 cm

Color.—upper surface at first opening: Near 154B upper surface at maturity: Near 155B upper surface at fading: Near 155A under surface at first opening: Near 154A under surface at maturity: Near 155B under surface at fading: Near 154A.

Disc florets:

Appearance.—Shiny

Texture.—Smooth.

Average number per flower.—Approximately 1

Shape.—Cylindric.

Apex.—Obtuse.

Average length.—Approximately 0.3 cm.

Average width.—Approximately 0.1 cm.

Color.—at first opening: Near 154B at maturity: Near 154C at fading: Near 154C.

Peduncle:

Length.—at terminal end (shortest): Approximately 3.5 cm. at lateral end (longest): Approximately 6 cm.

Strength.—Moderate.

Color.—Near 147B.

Habit.—Erect.

Inflorescence bud:

Length.—Approximately 0.6 cm.

Diameter.—Approximately 0.6 cm.

Form.—Globular.

Color.—Near 154C.

Sepals:

Appearance.—Matte

Texture.—Lightly pubescent

Number.—Approximately 20

Shape.—Ovate

Margin.—Entire

Apex.—Acute

Color.—upper side: Near 137C under side: Near 137B

### Reproductive Organs

Ray Florets:

Pistillate.—Number of pistils per flower: 1 Stigma shape: 2 branched Stigma color: Near 12A Stigma length: Approximately 0.1 cm Style color: Near 154C Style length: Approximately 0.2 cm.

Disc Florets:

Pistillate.—Number of pistils per flower: 1 Stigma shape: Cylindric Style length: Approximately 0.5 cm

Staminate.—Number of stamens per flower: Approximately 5 Anther shape: Tubular Anther color: Near 12A

Pollen color.—Near 12A

### Other Characteristics

Disease Resistance: Neither resistance nor susceptibility observed

What is claimed is:

1. A new and distinct variety of Chrysanthemum plant named ‘GEDI ONE CEL’ as herein illustrated and described.
